SEQUENCE LISTING (17 sequences)

SEQ ID NO: 1 - DNA, 2058 bp, Homo sapien
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 2 - protein, 685 aa, Homo sapien
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 3 - DNA, 3427 bp, Homo sapien
ctcgcaggctaggaacccgaggccaagagctgcagccaaagtcacttgggtgcagtgtactccctcactagcccgctcgagaccctaggatttgctccaggacacgtacttagagcagccaccgcccagtcgccctcacctggattacctaccgaggcatcgagcagcggagtttttgagaaggcgacaagggagcagcgtcccgaggggaatcagcttttcaggaactcggctggcagacgggacttgcgggagagcgacatccctaacaagcagattcggagtcccggagtggagaggacaccccaagggatgacgcctgcgtcccggagcgcctgtcgctgggcgctactgctgctggcggtactgtggccgcagcagcgcgctgcgggctccggcatcttccagctgcggctgcaggagttcgtcaaccagcgcggtatgctggccaatgggcagtcctgcgaaccgggctgccggactttcttccgcatttgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgtgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgcctgaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggcaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctgctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcagcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggtataaggcaggagcctactcagacacccagctccggcccagcagctgggccttccttctgcattgtttacattgcatcctgtatgggacatctttagtatgcacagtgctgctctgcggaggaggagggaatggcatgaactgaacagactgtgaacccgccaagagttgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagcaggtggggtgccacctgcagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttttgggcccttgagcctgggctccaccagtgcccactgttgccccgagaccaaccttgaagccgattttcaaaaatcaataatatgaggttttgttttgtagtttattttggaatctagtattttgataatttaagaatcagaagcactggcctttctacattttataacattattttgtatataatgtgtatttataatatgaaacagatgtgtacataaaaaaaaaaaaaaaaaaaaaaaaaaaaaa

SEQ ID NO: 4 - protein, 686 aa, Homo sapien
MTPASRSACRWALLLLAVLWPQQRAAGSGIFQLRLQEFVNQRGMLANGQSCEPGCRTFFRICLKHFQATFSEGPCTFGNVSTPVLGTNSFVVRDKNSGSGRNPLQLPFNFTWPGTFSLNIQAWHTPGDDLRPETSPGNSLISQIIIQGSLAVGKIWRTDEQNDTLTRLSYSYRVICSDNYYGESCSRLCKKRDDHFGHYECQPDGSLSCLPGWTGKYCDQPICLSGCHEQNGYCSKPDECICRPGWQGRLCNECIPHNGCRHGTCSIPWQCACDEGWGGLFCDQDLNYCTHHSPCKNGSTCSNSGPKGYTCTCLPGYTGEHCELGLSKCASNPCRNGGSCKDQENSYHCLCPPGYYGQHCEHSTLTCADSPCFNGGSCRERNQGSSYACECPPNFTGSNCEKKVDRCTSNPCANGGQCLNRGPSRTCRCRPGFTGTHCELHISDCARSPCAHGGTCHDLENGPVCTCPAGFSGRRCEVRITHDACASGPCFNGATCYTGLSPNNFVCNCPYGFVGSRCEFPVGLPPSFPWVAVSLGVGLVVLLVLLVMVVVAVRQLRLRRPDDESREAMNNLSDFQKDNLIPAAQLKNTNQKKELEVDCGLDKSNCGKLQNHTLDYNLAPGLLGRGSMPGKYPHSDKSLGEKVPLRLHSEKPECRISAICSPRDSMYQSVCLISEERNECVIATEV

SEQ ID NO: 5 - DNA, 9312 bp, Homo sapien
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagcagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaaacggcgcgccccacgagaccccggcttcctttcccaagccttcgggcgtctgtgtgcgctctgtggatgccagggccgaccagaggagcctttttaaaacacatgtttttatacaaaataagaacgaggattttaattttttttagtatttatttatgtacttttattttacacagaaacactgcctttttatttatatgtactgttttatctggccccaggtagaaacttttatctattctgagaaaacaagcaagttctgagagccagggttttcctacgtaggatgaaaagattcttctgtgtttataaaatataaacaaagattcatgatttataaatgccatttatttattgattccttttttcaaaatccaaaaagaaatgatgttggagaagggaagttgaacgagcatagtccaaaaagctcctggggcgtccaggccgcgccctttccccgacgcccacccaaccccaagccagcccggccgctccaccagcatcacctgcctgttaggagaagctgcatccagaggcaaacggaggcaaagctggctcaccttccgcacgcggattaatttgcatctgaaataggaaacaagtgaaagcatatgggttagatgttgccatgtgttttagatggtttcttgcaagcatgcttgtgaaaatgtgttctcggagtgtgtatgccaagagtgcacccatggtaccaatcatgaatctttgtttcaggttcagtattatgtagttgttcgttggttatacaagttcttggtccctccagaaccaccccggccccctgcccgttcttgaaatgtaggcatcatgcatgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctgtaaatatgtttttaaaggtggattttgtttaaaaaatcttaatgaatgagtctgttgtgtgtcatgccagtgagggacgtcagacttggctcagctcggggagccttagccgcccatgcactggggacgctccgctgccgtgccgcctgcactcctcagggcagcctcccccggctctacgggggccgcgtggtgccatccccagggggcatgaccagatgcgtcccaagatgttgatttttactgtgttttataaaatagagtgtagtttacagaaaaagactttaaaagtgatctacatgaggaactgtagatgatgtatttttttcatcttttttgttaactgatttgcaataaaaatgatactgatggtgaaaaaaaaaaaaaaa

SEQ ID NO: 6 - protein, 2556 aa, Homo sapien
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQAGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 7 - DNA, 6009 bp, Homo sapien
atgcagcccccttcactgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacgtacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccgggctgtgaggtgaatccagacaactgtgtcagccaccaatgtcagaatgggggcacttgccaggatgggctggacacctacacctgcctctgcccagaaacctggacaggctgggactgctccgaagatgtggatgagtgtgaggcccagggtccccctcactgcagaaacgggggcacctgccagaactctgctggtagctttcactgcgtgtgtgtgagtggctgggggggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggcttccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacacagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaatag

SEQ ID NO: 8 - protein, 2003 aa, Homo sapien
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 9 - DNA, 19 bp, Homo sapien
gaggtccaagccgaacctg

SEQ ID NO: 10 - DNA, 21 bp, Homo sapien
atcgctgatgtgcagttcaca

SEQ ID NO: 11 - DNA, 20 bp, Homo sapien
cgctgccggcctggattcac

SEQ ID NO: 12 - DNA, 17 bp, Homo sapien
caagcccggaagaagcg

SEQ ID NO: 13 - DNA, 20 bp, Homo sapien
tcgtcgcaattcagaaaggc

SEQ ID NO: 14 - DNA, 21 bp, Homo sapien
aacggcgcagagaccgcatca

SEQ ID NO: 15 - DNA, 5862 bp, Homo sapien
atgccacggctcctgacgcccttgctctgcctaacgctgctgcccgcgctcgccgcaagaggcttgagatgctcccagccaagtgggacctgcctgaatggaggtaggtgcgaagtggccagcggcactgaagcctgtgtctgcagcggagcctttgtgggccaacgatgccaggactccaatccttgcctcagcacaccgtgtaagaatgctggaacgtgccacgttgtggaccatggtggcactgtggattatgcctgcagctgtcccctgggtttctctgggcccctctgcctgacacctctggacaacgcctgcctggccaacccctgccgcaatgggggcacctgtgacctgctcactctcacagagtacaagtgccgctgcccaccagggtggtcaggaaaatcatgtcagcaggctgacccctgtgcctccaacccctgtgccaatggtggccagtgcctgccctttgagtcttcatacatctgtcgctgcccgcctggcttccatggccccacctgcaggcaagatgttaatgagtgcagccagaaccctgggctgtgccgccatggaggcacctgccacaatgagatcggctcctatcgctgtgcctgccgtgccacccatactggtccccactgtgaactgccctatgtgccctgcagcccctcaccctgccagaatggaggcacctgccgtcctacaggggacaccacccacgagtgtgcctgcttgccaggttttgctggacagaactgtgaagaaaatgtggatgactgtccaggaaacaactgcaagaatgggggtgcctgtgtggacggcgtgaatacctacaattgccgctgcccaccggagtggacgggtcagtactgtacagaggatgtggacgaatgtcagctcatgcccaatgcctgccagaatggcggaacctgccacaacacacacggcggctacaactgtgtgtgtgtcaatgggtggactggcgaggactgcagtgagaacattgatgactgtgccagtgccgcctgtttccagggtgccacttgccacgaccgtgtggcttccttctactgcgaatgtccgcatgggcgcacaggtctgctgtgccacctcaacgatgcgtgcatcagcaacccctgcaacgagggctccaactgtgacaccaaccctgtcaacggcaaagccatctgcacctgcccctcggggtacacagggccagcctgcagccaggacgtggatgagtgtgctctgggtgccaacccttgtgagcacgcaggcaaatgcctcaacacactgggttcttttgagtgccagtgtctacagggctacacgggaccccgctgtgagattgatgttaatgagtgcatctccaacccatgtcagaatgatgccacttgcctggaccagattggggagttccaatgcatatgtatgccaggttatgaaggtgtatactgtgaaatcaacacggatgagtgcgccagcagcccctgtctgcacaatggccactgcatggacaagatcaatgagttccaatgtcagtgccccaaaggcttcaacgggcacctgtgccagtatgatgtggatgagtgtgccagcacaccatgcaagaacggtgccaagtgcctggatgggcccaacacctatacctgcgtgtgtacagaaggttacacagggacccactgcgaagtggacattgacgagtgtgaccctgacccctgccactatggttcctgtaaggatggtgtggccacctttacctgcctgtgccagccaggctacacaggccatcactgtgagaccaacatcaatgagtgccacagccaaccgtgccgccatgggggcacctgccaggaccgtgacaactcctacctctgcttatgcctcaagggaaccacagggcccaactgtgagatcaacctggatgactgcgccagcaacccctgtgactctggcacctgtctggacaagattgatggctacgaatgtgcctgtgaaccaggctacacaggaagcatgtgtaacgtcaacattgacgaatgtgcgggcagcccctgccacaacgggggcacttgtgaggatggcatcgcgggcttcacttgccgctgccccgagggctaccatgaccccacgtgcctgtccgaggtcaacgagtgcaacagtaacccctgcatccacggagcttgccgggatggcctcaatgggtacaagtgtgactgtgcccctgggtggagtggaacaaactgtgacatcaacaacaacgagtgtgagtccaacccttgtgtcaacggtggcacctgcaaggacatgaccagtggctacgtatgcacctgccgagaaggcttcagtggccctaattgccagaccaacatcaacgaatgtgcctccaacccctgcctgaaccaggggacctgcattgatgatgtcgctggatacaagtgcaactgtcctctgccatatacaggagccacgtgtgaggtggtgttggccccatgtgctaccagcccctgcaaaaacagcggggtatgcaaggagtctgaagactatgagagtttttcctgtgtctgtcccacaggctggcaaggtcaaacctgcgaggttgacatcaatgagtgtgtgaaaagcccatgtcgccatggggcctcctgccagaacaccaatggcagctaccgctgcctctgccaggccggctatacaggtcgcaactgtgagagtgacatcgatgactgccgccccaacccgtgtcacaatgggggttcctgcaccgatggcatcaacacagccttctgcgactgcctgcccggcttccagggtgccttctgtgaggaggacatcaatgaatgtgccagcaatccctgccaaaatggtgccaattgcactgactgtgtggacagctacacatgtacctgccccgtgggcttcaatggcatccactgcgagaacaacacacctgactgtactgagagctcctgcttcaatggtggtacctgtgtggatggtatcaactccttcacctgtctgtgtccacctggcttcacgggcagctactgtcagtatgatgtcaatgagtgtgattcacggccctgtctgcacggtggtacctgccaagacagctatggtacttataagtgtacctgcccacagggctacactggtctcaactgccagaaccttgtgcgctggtgcgactcggctccctgcaagaatggtggcaggtgctggcagaccaacacgcagtaccactgtgagtgccgcagcggctggactggcgtcaactgcgacgtgctcagtgtgtcctgtgaggtggctgcacagaagcgaggcattgacgtcactctcctgtgccagcatggagggctctgtgtggatgagggagataaacattactgccactgccaggcaggctacacgggcagctactgtgaggacgaggtggacgagtgctcacctaacccctgccagaatggagctacctgcactgactatctcggcggcttttcctgcaagtgtgtggctggctaccatgggtctaactgctcagaggagatcaacgagtgcctgtcccagccctgccagaatgggggtacctgcattgatctgaccaactcctacaagtgttcctgcccccgggggacacagggtgtacactgtgagatcaatgttgatgactgccatcccccccttgaccctgcctcccgaagccccaagtgcttcaacaatggcacctgtgtggaccaggtgggtggctatacctgcacctgcccaccaggcttcgtcggggagcggtgtgagggtgatgtcaatgaatgtctctccaacccctgtgacccacgtggcacccagaactgtgtgcagcgtgttaatgacttccactgcgagtgccgggctggccacactggacgccgctgtgagtcagtcatcaatggctgcaggggcaaaccttgcaagaatgggggtgtctgtgccgtggcctccaacaccgcccgtggattcatctgtaggtgccctgcgggcttcgagggtgccacatgtgagaatgatgcccgcacttgtggcagcttacgctgcctcaacggtggtacatgcatctcgggcccacgtagtcccacctgcctatgcctgggatccttcaccggccctgagtgccagttcccagccagcagcccctgtgtgggtagcaacccctgctacaatcagggcacctgtgagcccacatccgagaaccctttctaccgctgtctatgccctgccaaattcaacgggctactgtgccacatcctggactacagcttcacaggtggcgctgggcgcgacattcccccaccgcagattgaggaggcctgtgagctgcctgagtgccaggtggatgcaggcaataaggtctgcaacctgcagtgtaataatcacgcatgtggctgggatggtggcgactgctccctcaacttcaatgacccctggaagaactgcacgcagtctctacagtgctggaagtattttagcgacggccactgtgacagccagtgcaactcggccggctgcctctttgatggcttcgactgccagctcaccgagggacagtgcaaccccctgtatgaccagtactgcaaggaccacttcagtgatggccactgcgaccagggctgtaacagtgccgaatgtgagtgggatggcctagactgtgctgagcatgtacccgagcggctggcagccggcaccctggtgctggtggtgctgcttccacccgaccagctacggaacaactccttccactttctgcgggagctcagccacgtgctgcacaccaacgtggtcttcaagcgtgatgcgcaaggccagcagatgatcttcccgtactatggccacgaggaagagctgcgcaagcacccaatcaagcgctctacagtgggttgggccacctcttcactgcttcctggtaccagtggtgggcgccagcgcagggagctggaccccatggacatccgtggctccattgtctacctggagatcgacaaccggcaatgtgtgcagtcatcctcgcagtgcttccagagtgccaccgatgtggctgccttcctaggtgctcttgcgtcacttggcagcctcaatattccttacaagattgaggccgtgaagagtgagccggtggagcctccgctgccctcgcagggcccgggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 16 - protein, 1953 aa, Homo sapien
MPRLLTPLLCLTLLPALAARGLRCSQPSGTCLNGGRCEVASGTEACVCSGAFVGQRCQDSNPCLSTPCKNAGTCHVVDHGGTVDYACSCPLGFSGPLCLTPLDNACLANPCRNGGTCDLLTLTEYKCRCPPGWSGKSCQQADPCASNPCANGGQCLPFESSYICRCPPGFHGPTCRQDVNECSQNPGLCRHGGTCHNEIGSYRCACRATHTGPHCELPYVPCSPSPCQNGGTCRPTGDTTHECACLPGFAGQNCEENVDDCPGNNCKNGGACVDGVNTYNCRCPPEWTGQYCTEDVDECQLMPNACQNGGTCHNTHGGYNCVCVNGWTGEDCSENIDDCASAACFQGATCHDRVASFYCECPHGRTGLLCHLNDACISNPCNEGSNCDTNPVNGKAICTCPSGYTGPACSQDVDECALGANPCEHAGKCLNTLGSFECQCLQGYTGPRCEIDVNECISNPCQNDATCLDQIGEFQCICMPGYEGVYCEINTDECASSPCLHNGHCMDKINEFQCQCPKGFNGHLCQYDVDECASTPCKNGAKCLDGPNTYTCVCTEGYTGTHCEVDIDECDPDPCHYGSCKDGVATFTCLCQPGYTGHHCETNINECHSQPCRHGGTCQDRDNSYLCLCLKGTTGPNCEINLDDCASNPCDSGTCLDKIDGYECACEPGYTGSMCNVNIDECAGSPCHNGGTCEDGIAGFTCRCPEGYHDPTCLSEVNECNSNPCIHGACRDGLNGYKCDCAPGWSGTNCDINNNECESNPCVNGGTCKDMTSGYVCTCREGFSGPNCQTNINECASNPCLNQGTCIDDVAGYKCNCPLPYTGATCEVVLAPCATSPCKNSGVCKESEDYESFSCVCPTGWQGQTCEVDINECVKSPCRHGASCQNTNGSYRCLCQAGYTGRNCESDIDDCRPNPCHNGGSCTDGINTAFCDCLPGFQGAFCEEDINECASNPCQNGANCTDCVDSYTCTCPVGFNGIHCENNTPDCTESSCFNGGTCVDGINSFTCLCPPGFTGSYCQYDVNECDSRPCLHGGTCQDSYGTYKCTCPQGYTGLNCQNLVRWCDSAPCKNGGRCWQTNTQYHCECRSGWTGVNCDVLSVSCEVAAQKRGIDVTLLCQHGGLCVDEGDKHYCHCQAGYTGSYCEDEVDECSPNPCQNGATCTDYLGGFSCKCVAGYHGSNCSEEINECLSQPCQNGGTCIDLTNSYKCSCPRGTQGVHCEINVDDCHPPLDPASRSPKCFNNGTCVDQVGGYTCTCPPGFVGERCEGDVNECLSNPCDPRGTQNCVQRVNDFHCECRAGHTGRRCESVINGCRGKPCKNGGVCAVASNTARGFICRCPAGFEGATCENDARTCGSLRCLNGGTCISGPRSPTCLCLGSFTGPECQFPASSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQGPGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 5862 bp, Homo sapien
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagcagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagggcccgggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2006/0134121 A1
(43) Pub. Date: Jun. 22, 2006

(54) DII4 antagonists, assays, and therapeutic methods thereof

(76) Inventors: Gavin Thurston, White Plains, NY (US); Nicholas Gale, Yorktown Heights, NY (US); Irene Noguera, Hastings-on-Hudson, NY (US)

Correspondence Address: REGENERON PHARMACEUTICALS, INC, 777 OLD SAW MILL RIVER ROAD, TARRYTOWN, NY, 10591, US

(21) Appl. No.: 11/264,243

(22) Filed: Oct. 31, 2005

Related U.S. Application Data
(60) Provisional application No. 60/623,658, filed on Oct. 29, 2004.

(51) Int. Cl. A61K 48/00; G01N 33/574; A61K 39/395; C07K 16/30

(52) U.S. Cl. 424/155.1; 435/7.23; 514/44; 530/388.8

(57) ABSTRACT

Methods for screening for agents capable of inhibiting Dll4 are provided, as well as therapeutic methods for treating Dll4-mediated conditions. More specifically, methods are provided for identifying agents capable of inhibiting blood vessel growth and formation, such as antibodies to human DLL4.

### CROSS-REFERENCE TO RELATED APPLICATIONS

This application claims the benefit under 35 USC § 119(e) of U.S. Provisional application 60/623,658 filed 29 Oct. 2004, which application is herein specifically incorporated by reference in its entirety.

### BACKGROUND OF THE INVENTION

1. Field of the Invention

This invention is related to Dll4, a member of the Delta family of Notch ligands, screening assays for identifying inhibitors of Dll4, Dll4 antagonists, and therapeutic methods using such compounds.

2. Description of Related Art

The Notch-signaling pathway is a system for cell-to-cell communication used by a wide range of eukaryotes for many biological processes, such as differentiation, proliferation, and homeostasis. Delta like 4 (Dl4) or delta-like ligand 4 (Dll4) (hereinafter “Dll4”) is a member of the Delta family of Notch ligands which exhibits highly selective expression by vascular endothelium (Shutter et al. (2000) Genes Develop. 14:1313-1318). Dll4 is a ligand for Notch1 and Notch 4 receptors. The nucleic acid encoding human (SEQ ID NO:1) and mouse Dll4 (SEQ ID NO:3), as well as the human (SEQ ID NO:2) and mouse (SEQ ID NO:4) proteins are shown in FIGS. 1 and 2. Gene targeted Dll4 mice have been generated (Duarte et al. (2004) Genes & Dev. 18: doi: 10.1101/gad.1239004; Krebs et al. (2004) Genes & Dev. 18: doi: 10.1101/gad.1239204: Gale et al. (2004) Proc Natl Acad Sci USA 101: 15949-15954).

### BRIEF SUMMARY OF THE INVENTION

This invention is based in part on the observation that the expression of Dll4 is up-regulated in tumors over-expressing vascular endothelial growth factor (VEGF), and is down-regulated with exposure to a VEGF antagonist.

In a first aspect, the invention features screening methods for identifying agents capable of binding Dll4. The screening methods of the invention include in vitro and in vivo assays. Examples of agents to be tested by the screening methods of the invention include, but are not limited to, nucleic acids (e.g., DNA and RNA), carbohydrates, lipids, proteins, peptides, peptidomimetics, small molecules and other compounds. Agents can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

In one embodiment of an in vitro screening method of the invention, agents capable of binding Dll4 are identified in a cell-based assay system. More specifically, cells expressing a Dll4 protein or a Dll4 fragment, are contacted with a test compound or a control compound, and the ability of the candidate compound to bind Dll4 or a fragment thereof is determined. In a more specific competitive binding embodiment, the test compound is contacted with the cell in the presence of a Dll4 ligand, and the ability of the test compound to bind Dll4 in the presence of the competitive Dll4 ligand is determined. In an even more specific embodiment, the Dll4 ligand is labeled. Labeling of the Dll4 ligand may be performed by any method known to the art, including for example, radioactivity or fluorescence.

In another embodiment, agents capable of binding a Dll4 protein or protein fragment are identified in a cell-free assay system. More specifically, a native or recombinant human Dll4 protein or protein fragment is contacted with a candidate compound or a control compound, and the ability of the candidate compound to bind Dll4 or a fragment thereof is determined.

In a second aspect, the invention features screening methods for identifying agents capable of inhibiting Dll4 activity or expression. The screening methods of the invention include in vitro and in vivo assays. In one embodiment, the agent capable of inhibiting Dll4 is an antagonist to a natural Dll4 ligand capable of binding to human Dll4. In a more specific embodiment, the antagonist is an antibody, more specifically, a blocking antibody. The antibody may be polyclonal, monoclonal, chimeric, humanized, or a wholly human antibody. In another in vitro embodiment, the ability of an agent to inhibit the binding of Dll4 to the Notch1 or Notch 4 receptor are tested in an assay system comprising a Notch1 or Notch4 protein, and the ability of Dll4 to bind its receptor is determined in the presence and absence of a test agent. An inhibitor of Dll4 activity includes an agent capable of blocking the binding of Dll4 to its receptor, and may include for example, an antibody, a small molecule, or a modified Dll4 molecule capable of binding, but not activating its receptor. In another embodiment, the agent capable of inhibiting Dll4 expression is an antisense molecule, a ribozyme or triple helix, or a short interfering RNA (siRNA) capable of silencing Dll4 gene expression.

In a third aspect, the invention features a method of treating a Dll4-mediated condition, comprising administering an agent capable of inhibiting Dll4 activity or expression. The agent may be an antagonist, such as a blocking antibody, a modified Dll4 molecule which binds but does not activate its Notch receptor, an antisense or siRNA molecule, or an agent identified by the method of the invention. The Dll4-mediated condition is a condition in which it is desirable to inhibit blood vessel growth or development.

In a fourth aspect, the invention features Dll4 antagonists capable of binding and inhibiting Dll4. In one embodiment, the Dll4 antagonist of the invention is a fusion protein comprising at least one soluble Notch receptor or fragment thereof capable of binding Dll4, fused to a multimerizing component. In specific embodiments, the soluble Notch receptor is human Notch 1 (SEQ ID NO:5-6) or Notch 4 (SEQ ID NO:7-8). The multimerizing component may be any component capable of forming a higher order complex through interaction with a multimerizing component on a different fusion protein.

In specific embodiments wherein the multimerizing component, may be selected from the group consisting of (i) a multimerizing component comprising a cleavable region (C-region), (ii) a truncated multimerizing component, (iii) an amino acid sequence between 1 to about 500 amino acids in length, optionally comprising at least one cysteine residue, (iv) a leucine zipper, (v) a helix loop motif, (vi) a coil-coil motif, (vii) an Fc-protein, and (viii) a combination thereof.

The fusion protein may optionally comprise a signal sequence, which may comprise any sequence known to a skilled artisan for directing secretion of a polypeptide or protein from a cell, include natural or synthetic sequences. Generally, a signal sequence is placed at the beginning or amino-terminus of the fusion protein of the invention. Such a signal sequence may be native to the cell, recombinant, or synthetic.

The components of the fusion protein of the invention may be connected directly to each other or connected via one or more spacer sequences. In one preferred embodiment, the components are fused directly to each other. In another preferred embodiment, the components are connected with a nucleic acid sequence encoding a spacer of 1-200 amino acids. Any spacer known to the art may be used to connect the protein components. A spacer sequence may also include a sequence used to enhance expression of the fusion protein, provide restriction sites, and allow component domains to form optimal tertiary and quaternary structures and/or to enhance the interaction of a component with its receptor. In one embodiment, the fusion protein of the invention comprises one or more peptide sequences between one or more components that is (are) between 1-25 amino acids.

The components of the fusion protein of the invention may be arranged in a variety of configurations. For example, the soluble receptor component (1), and the multimerizing component (2) may be arranged in one of the following configurations: 1-2; 2-1; 1-1-2; 1-2-1, 2-1-1.

In a fifth aspect, the invention features pharmaceutical compositions useful for inhibition of blood vessel growth or development, comprising an agent capable of inhibiting Dll4 activity or expression. In one embodiment, an agent is one that was identified by a screening method of the invention. In another embodiment, the agent is a blocking antibody. In another embodiment, the molecule is a modified Dll4 polypeptide which is capable of binding its Notch receptor but such binding does not result in activation of the receptor. In yet another embodiment, the agent is a nucleic acid capable of interfering with the expression of Dll4. In a preferred embodiment, the pharmaceutical composition comprises a fusion protein of the invention and a pharmaceutically acceptable carrier.

The invention features an antibody or an antibody fragment or antibody-like molecule capable of binding and inhibiting the human Dll4 protein. In a specific embodiment, the antibody is a human antibody and is useful as a therapeutic to treat tumor angiogenesis and other pathological angiogenesis.

Other objects and advantages will become apparent from a review of the ensuing detailed description.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

Definitions

By the term “Dll4-associated” or “Dll4-mediated” condition or disease is meant a condition which is affected directly or indirectly by modulation of Dll4 activity. More specifically, Dll4 is now shown to be involved in blood vessel growth and development. Accordingly, in one embodiment, a Dll4-associated condition treatable by the method of the invention is one in which it is desirable to inhibit or reduce Dll4-mediated blood vessel growth or development, e.g., to inhibit tumor development.

By the term “inhibitor” is meant a substance which retards or prevents a chemical or physiological reaction or response. Inhibition of Dll4 activity may be direct, through inhibition of receptor activation with a blocking antibody, for example, or indirect, resulting from interference with expression of the gene encoding Dll4. Common inhibitors include but are not limited to antisense molecules, antibodies, soluble receptors, antagonists and their derivatives, and modified Dll4 ligands which bind their Notch receptor but are unable to activate signaling through such binding.

A “knock-out” animal is an animal generated from a mammalian cell which carries a genetic modification resulting from the insertion of a DNA construct targeted to a predetermined, specific chromosomal location which alters the function and/or expression of a gene that was at the site of the targeted chromosomal location. In both cases, the DNA construct may encode a reporter protein such as lacZ, protein tags, and proteins, including recombinases such as Cre and FLP. A “knock-in” animal is an animal generated from a mammalian cell which carries a genetic modification resulting from the insertion of a DNA construct targeted to a predetermined, specific chromosomal location which may or may not alter the function and/or expression of the gene at the site of the targeted chromosomal location.

General Description

This invention is based in part on elucidation of the function of Dll4 as involved in the development and growth of blood vessels. Accordingly, these discoveries provide new methods for the treatment of Dll4-mediated conditions, by allowing the identification and design of agents capable of inhibiting Dll4 activity or expression.

Screening Assays

The present invention provides methods for identifying agents (e.g., candidate compounds or test compounds) that are capable of inhibiting Dll4 activity or Dll4-mediated blood vessel growth and/or development. Agents identified through the screening method of the invention are potential therapeutics for use in inhibiting blood vessel development and/or growth in conditions where that development or growth is undesirable, e.g., blood vessel development and growth associated with disease such as tumor formation.

Examples of agents include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

In one embodiment, agents that bind Dll4 are identified in a cell-based assay system. In accordance with this embodiment, cells expressing a Dll4 protein or protein fragment are contacted with a candidate (or a control compound), and the ability of the candidate compound to bind Dll4 is determined. The cell may be of prokaryotic origin (e.g., E. coli) or eukaryotic origin (e.g., yeast or mammalian). In specific embodiments, the cell is a Dll4 expressing mammalian cell, such as, for example, a COS-7 cell, a 293 human embryonic kidney cell, a NIH 3T3 cell, or Chinese hamster ovary (CHO) cell. Further, the cells may express a Dll4 protein or protein fragment endogenously or be genetically engineered to express a Dll4 protein or protein fragment. In some embodiments of the binding assays of the invention, the compound to be tested may be labeled. Cells expressing the Dll4 receptor are then incubated with labeled test compounds, in binding buffer, in cell culture dishes. To determine non-specific binding, unlabeled ligand may be added to the wells. After the incubation, bound and free ligands are separated and detection activity measured in each well.

In specific embodiments, the cell-based assay system may measure the ability of Dll4 to bind the Notch 1 or Notch 4 receptor in the presence of the test agent. A desirable assay cell may express the Notch receptor or a fragment of a Notch receptor capable of binding by Dll4. Detection of bound and free ligand may be determined as described above or by any method known to the art.

The assay methods of the invention are useful to identify agents that inhibit Dll4 activity. The ability of the candidate compound to alter the activity of Dll4 can be determined by methods known to those of skill in the art, for example, by flow cytometry, a scintillation assay, immunoprecipitation or western blot analysis. For example, modulators of Dll4 activity may be identified using a biological readout in cells expressing a Notch 1 or Notch 4 receptor protein. Antagonists are identified by incubating cells or cell fragments expressing Dll4 with test compound and measuring a biological response in these cells and in parallel cells or cell fragments not expressing Dll4. An increased biological response in the cells or cell fragments expressing Dll4 compared to the parallel cells or cell fragments indicates the presence of an agonist in the test sample, whereas a decreased biological response indicates an antagonist.

In more specific embodiments, detection of binding and/or inhibition of a test agent to a Dll4 protein may be accomplished by detecting a biological response, such as, for example, measuring Ca²⁺ ion flux, cAMP, IP₃, PIP₃ and transcription of reporter genes. For example, to identify ligands of Dll4, cells expressing the receptor may be screened against a panel of know compounds utilizing a bioluminescent signal such as the aequorin luminescence assays (see, for example, Button et al. (1993) Cell. Calcium 14:663-671; Liu et al. (1999) Biochem. Biophys. Res. Comm. 266:174-178; Ungrin et al. (1999) Anal. Biochem. 272:34-42; Fujii et al. (2000) J. Biol. Chem 275:21086-21074; Raddatz et al. (2000) J. Biol. Chem. 275:32452-32459; and Shan et al. (2000) J. Biol. Chem. 275:39482-39486, which references are herein specifically incorporated by reference in their entireties). Suitable reporter genes include endogenous genes as well as exogenous genes that are introduced into a cell by any of the standard methods familiar to the skilled artisan, such as transfection, electroporation, lipofection and viral infection. The invention further includes other end point assays to identify compounds that inhibit receptor activity, such as those associated with signal transduction. When the cells are tumor tissue, the biological assay of Dll4 activity may include measure of blood vessel development.

In another embodiment, agents that inhibit Dll4-mediated activity are identified in a cell-free assay system. In accordance with this embodiment, a Dll4 protein or protein fragment is contacted with a test (or control) compound and the ability of the test compound to bind Dll4 is determined. Competitive binding may also be determined in the presence of a Notch 1 or Notch 4 receptor protein. In vitro binding assays employ a mixture of components including a Dll4 protein or protein fragment, which may be part of a fusion product with another peptide or polypeptide, e.g., a tag for detection or anchoring, and a sample suspected of containing a natural Dll4 binding target, e.g., a Notch 1 or Notch 4 receptor. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature which facilitates optimal binding. The mixture is incubated under conditions whereby the Dll4 protein binds the test compound. Incubation periods are chosen for optimal binding but are also minimized to facilitate rapid, high-throughput screening.

After incubation, the binding between the Dll4 protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radiative emissions, nonradiative energy transfers, or indirectly detected with antibody conjugates.

It may be desirable to immobilize either the receptor protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a fusion protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule, as well as enzyme-linked assays which rely on detecting an enzymatic activity associated with the target molecule.

In another embodiment, agents that inhibit Dll4 activity or expression are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the Dll4 activity or expression is determined.

Antibodies to Human Dll4 Protein and Ligands

According to the invention, a Dll4 protein, protein fragment, derivative or variant, may be used as an immunogen to generate immunospecific antibodies. Further, the present invention includes antibodies to compounds capable of binding Dll4 or capable of binding its target receptor. The present invention provides for an antibody or an antibody-like molecule which specifically binds human Dll4 and is useful to inhibit the development or growth of blood vessels. The term “antibody-like” molecule encompasses antagonist molecules containing one or more antibody fragments (e.g., a Dll4-specific ScFv) optionally fused to a multimerizing component, e.g., a “trap”-like molecule capable of binding and inhibiting Dll4. For a description of trap-like molecules, see U.S. Pat. No. 6,472,179 Stahl et al, herein specifically incorporated by reference in its entirety. Dll4 antagonists are further described below.

The Dll4 antagonists of the invention may comprise one or more immunoglobulin binding domains isolated from antibodies generated against human Dll4. The term “immunoglobulin or antibody” as used herein refers to a mammalian, including human, polypeptide comprising a framework region from an immunoglobulin gene or fragments thereof that specifically binds and recognizes an antigen, which, in the case of the present invention, is a Dll4 protein or portion thereof. If the intended antibody or antibody-like protein will be used as a mammalian therapeutic, immunoglobulin binding regions should be derived from the corresponding mammalian immunoglobulins. If the molecule is intended for non-therapeutic use, such as for diagnostics and ELISAs, the immunoglobulin binding regions may be derived from either human or non-human mammals, such as mice. The human immunoglobulin genes or gene fragments include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG₁, IgG₂, etc.) as well as allotypes thereof. Typically, the antigen-binding region of an antibody will be the most critical in determining specificity and affinity of binding.

An exemplary immunoglobulin (antibody) structural unit of human IgG, comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}-C_{H} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the terms antibody, as used herein, also includes antibody fragments either produced by the modification of whole antibodies, or those synthesized de novo using recombinant DNA methodologies (e.g., single chain Fv (scFv) single variable domains (Dabs)) or those identified using display libraries such as phage, E. coli or yeast display libraries (see, for example, McCafferty et al. (1990) Nature 348:552-554).

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). Antibodies that are isolated from organisms other than humans, such as mice, rats, rabbits, cows, can be made more human-like through chimerization or humanization.

“Humanized” or chimeric forms of non-human (e.g., murine) antibodies are immunoglobulins, immunoglobulin chains or fragments thereof (such as Fv, Fab, Fab′, F(ab′)2 or other antigen-binding subsequences of antibodies) that contain minimal sequences required for antigen binding derived from non-human immunoglobulin. They have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotype IgG1 is preferred. A typical chimeric antibody is thus a hybrid protein consisting of the V or antigen-binding domain from a mouse antibody and the C or effector domain from a human antibody. Humanized antibodies have variable region framework residues substantially from a human antibody (termed an acceptor antibody) and complementarity determining regions (CDR regions) substantially from a mouse antibody, (referred to as the donor immunoglobulin). See, Queen et al., Proc. Natl. Acad. Sci. USA 86:10029-10033 (1989) and WO 90/07861, U.S. Pat. Nos. 5,693,762, 5,693,761, 5,585,089, 5,530,101 and 5,225,539. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids. For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly; (2) is adjacent to a CDR region; (3) otherwise interacts with a CDR region (e.g. is within about 6 A of a CDR region), or (4) participates in the V_{L}-V_{H} interface. Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

Fully human antibodies may be made by any method known to the art. For example, U.S. Pat. No. 6,596,541 describes a method of generating fully human antibodies. Briefly, initially a transgenic animal such as a mouse is generated that produces hybrid antibodies containing human variable regions (VDJ/VJ) and mouse constant regions. This is accomplished by a direct, in situ replacement of the mouse variable region (VDJ/VJ) genes with their human counterparts. The mouse is then exposed to human antigen, or an immunogenic fragment thereof. The resultant hybrid immunoglobulin loci will undergo the natural process of rearrangements during B-cell development to produce hybrid antibodies having the desired specificity. The antibody of the invention is selected as described above. Subsequently, fully human antibodies are made by replacing the mouse constant regions with the desired human counterparts. Fully human antibodies can also be isolated from mice or other transgenic animals such as cows that express human transgenes or minichromosomes for the heavy and light chain loci. (Green (1999) J Immunol Methods. 231:11-23 and Ishida et al (2002) Cloning Stem Cells. 4:91-102) Fully human antibodies can also be isolated from humans to whom the protein has been administered. Fully human antibodies can also be isolated from immune compromised mice whose immune systems have been regenerated by engraftment with human stem cells, splenocytes, or peripheral blood cells (Chamat et al (1999) J Infect Dis. 180:268-77). To enhance the immune response to the protein of interest one can knockout the gene encoding the protein of interest in the human-antibody-transgenic animal.

The genes encoding the heavy and light chains of an antibody of interest can be cloned from a cell, either directly from the producing B cells from the blood, lymph node, spleen, etc or from hybridomas made from the B cells or from EBV immortalized B cells using standard technologies. Techniques for the production of single chain antibodies or recombinant antibodies (U.S. Pat. Nos. 4,946,778 and 4,816,567) can be adapted to produce antibodies used in the fusion proteins and methods of the instant invention. Also, transgenic mice, or other organisms such as other mammals, may be used to express human or humanized antibodies.

Alternatively, phage display or related display technologies can be used to identify antibodies, antibody fragments, such as variable domains, and heteromeric Fab fragments that specifically bind to selected antigens. Gene libraries encoding heavy and light chains of monoclonal antibodies can be made from the hybridoma, spleen, lymph node or plasma cells described above or from naïve, vaccinated, or diseased human sources of B cells. Random combinations of the heavy and light chain gene products generate a large pool of antibodies with different antigenic specificity. Phage display is of particular value to isolate weakly binding antibodies or fragments thereof from un-immunized animals which, when combined with other weak binders in accordance with the invention described herein, create strongly binding trapbodies.

Screening and selection of preferred immunoglobulins (antibodies) can be conducted by a variety of methods known to the art. Initial screening for the presence of monoclonal antibodies specific to Dll4 may be conducted through the use of ELISA-based methods or phage display, for example. A secondary screen is preferably conducted to identify and select a desired monoclonal antibody for use in construction of the trapbodies of the invention. Secondary screening may be conducted with any suitable method known to the art. One preferred method, termed “Biosensor Modification-Assisted Profiling” (“BiaMAP”) is described in U.S. Patent application 2004/101920, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody: antigen interactions. Alternatively, ELISA-based, bead-based, or Biacore-based competition assays can be used to identify binding pairs that bind different epitopes of Dll4 and thus are likely to cooperate to bind the ligand with high affinity.

Inhibitory Nucleic Acids

In addition to agents capable of inhibiting Dll4 activity, the methods of the invention encompass inhibition of Dll4 expression with nucleic acid molecules capable of interfering with or silencing Dll4 gene expression. In one embodiment, Dll4 expression is inhibited by Dll4 antisense nucleic acid comprises at least 6 to 200 nucleotides that are antisense to a gene or cDNA encoding Dll4 or a portion thereof. As used herein, a Dll4 “antisense” nucleic acid refers to a nucleic acid capable of hybridizing by virtue of some sequence complementarity to a portion of an RNA (preferably mRNA) encoding Dll4. The antisense nucleic acid may be complementary to a coding and/or noncoding region of an mRNA encoding Dll4. The oligonucleotides can be DNA or RNA or chimeric mixtures or derivatives or modified versions thereof, can be single- or double-stranded, and can be modified at the base moiety, sugar moiety, or phosphate backbone. The oligonucleotide may include other appended groups such as peptides, agents that facilitate transport across the cell membrane (see, e.g., Letsinger et al. (1989) Proc. Natl. Acad. Sci. USA 86:6553-6556) or blood-brain barrier (see, e.g., WO 89/10134). Such antisense nucleic acids have utility as compounds that inhibit Dll4 expression, and can be used in the treatment of undesirable blood vessel formation.

In another embodiment, Dll4 may be inhibited with ribozymes or triple helix molecules which decrease Dll4 gene expression. Ribozyme molecules designed to catalytically cleave gene mRNA transcripts encoding Dll4 can be used to prevent translation of Dll4 mRNA and, therefore, expression of the gene product. (See, e.g., PCT International Publication WO90/11364). Alternatively, the endogenous expression of Dll4 can be reduced by targeting deoxyribonucleotide sequences complementary to the regulatory region of the gene (i.e., the gene promoter and/or enhancers) to form triple helical structures that prevent transcription of Dll4 in target cells in the body (see, for example, Helene et al. (1992) Ann. N.Y. Acad. Sci., 660, 27-36).

In another embodiment, Dll4 is inhibited by a short interfering RNA (siRNA) through RNA interference (RNAi) or post-transcriptional gene silencing (PTGS) (see, for example, Ketting et al. (2001) Genes Develop. 15:2654-2659). siRNA molecules can target homologous mRNA molecules for destruction by cleaving the mRNA molecule within the region spanned by the siRNA molecule. Accordingly, siRNAs capable of targeting and cleaving homologous Dll4 mRNA are useful for inhibiting undesirable blood vessel formation.

Dll4 Antagonists

In a preferred embodiment, the Dll4 antagonist of the invention is a fusion protein comprising at least one soluble Notch receptor component fused to a multimerizing component. The fusion protein antagonists of the invention are capable of binding and inhibiting the biological activity of Dll4. The ability of a fusion protein of the invention to inhibit Dll4 can be determined in vitro, for example, as described in the Example section below.

The soluble extracellular domain of a Notch receptor is composed of multiple EGF-like domains. Accordingly, the instant invention envisions using the full length extracellular domain as well as fragments of the full length extracellular domain which retain the capacity to bind Dll4.

In specific embodiments, the fusion proteins of the invention comprise a multimerizing component. A multimerizing component includes any natural or synthetic sequence capable of interacting with another multimerizing component to form a higher order structure, e.g., a dimer, a trimer, etc. The multimerizing component may be selected from the group consisting of (i) a multimerizing component comprising a cleavable region (C-region), (ii) a truncated multimerizing component, (iii) an amino acid sequence between 1 to about 500 amino acids in length, (iv) a leucine zipper, (v) a helix loop motif, and (vi) a coil-coil motif. When the multimerizing component comprises an amino acid sequence between 1 to about 500 amino acids in length, the sequence may contain one or more cysteine residues capable of forming a disulfide bond with a corresponding cysteine residue on another fusion protein comprising a multimerizing component with one or more cysteine residues. In some embodiments, the multimerizing component comprises an immunoglobulin-derived domain from, for example, human IgG, IgM or IgA, or comparable immunoglobulin domains from other animals, including, but not limited to, mice. In specific embodiments, the immunoglobulin-derived domain may be selected from the group consisting of the constant region of IgG, the Fc domain of IgG, an Fc-protein and the heavy chain of IgG. The Fc domain of IgG may be selected from the isotypes IgG1, IgG2, IgG3, and IgG4, as well as any allotype within each isotype group.

The components of the fusion proteins of the invention may be connected directly to each other or be connected via spacers. The term “spacer” or “linker” means one or more molecules, e.g., nucleic acids or amino acids, or non-peptide moieties, such as polyethylene glycol, which may be inserted between one or more component domains. For example, spacer sequences may be used to provide a restriction site between components for ease of manipulation. A spacer may also be provided to enhance expression of the fusion protein from a host cell, to decrease steric hindrance such that the component may assume its optimal tertiary or quaternary structure and/or interact appropriately with its target molecule. For spacers and methods of identifying desirable spacers, see, for example, George et al. (2003) Protein Engineering 15:871-879, herein specifically incorporated by reference.

A spacer sequence may include one or more amino acids naturally connected to a receptor component, or may be an added sequence used to enhance expression of the fusion protein, provide specifically desired sites of interest, allow component domains to form optimal tertiary structures and/or to enhance the interaction of a component with its target molecule. In one embodiment, the spacer comprises one or more peptide sequences between one or more components which is (are) between 1-100 amino acids, preferably 1-25. In one specific embodiment, the spacer is a three amino acid sequence; more specifically, the three amino acid sequence of Gly Pro Gly.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. In a specific embodiment, the active agent is a fusion protein of the invention capable of inhibiting Dll4. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of a Dll4-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Combination Therapies

In numerous embodiments, the fusion proteins of the present invention may be administered in combination with one or more additional compounds or therapies. For example, multiple fusion proteins can be co-administered, or one or more fusion proteins can be administered in conjunction with one or more therapeutic compounds. In a preferred embodiment, the Dll4 inhibitor of the invention is administered with a VEGF antagonist, such as an anti-VEGF antibody or a VEGF trap. Preferred embodiments of a VEGF trap (as described in WO 00/75319, which publication is herein specifically incorporated by reference in its entirety) are selected from the group consisting of acetylated Flt-1(1-3)-Fc, Flt-1(1-3_{R->N})-Fc, Flt-1(1-3_{ΔB})-Fc, Flt-1(2-3_{ΔB})-Fc, Flt-1(2-3)-Fc, Flt-1D2-VEGFR3D3-FcΔC1(a), Flt-1D2-Flk-1D3-FcΔC1(a), and VEGFR1R2-FcΔC1(a).

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a knock-out or knock-in animal having a modified endogenous Dll4 gene. The invention contemplates a transgenic animal having an exogenous Dll4 gene generated by introduction of any Dll4-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the Dll4 protein to particular cells.

Knock-out animals containing a modified Dll4 gene as described herein are useful to identify Dll4 function. Methods for generating knock-out or knock-in animals by homologous recombination in ES cells are known to the art. Animals generated from ES cells by microinjection of ES cells into donor blastocytes to create a chimeric animal, which chimeric animal can be bred to produce an animal in which every cell contains the targeted modification. A transgenic animal can be produced by introducing nucleic acid into the male pronuclei of a fertilized oocyte, e.g., by microinjection, retroviral infection, and allowing the oocyte to develop in a pseudopregnant female foster animal. Further, random transgenic animals containing an exogenous Dll4 gene, e.g., a human Dll4 gene, may be useful in an in vivo context since various physiological factors that are present in vivo and that could effect ligand binding, Dll4 activation, and signal transduction, may not be evident from in vitro cell-free or cell-based assays. Accordingly, it is useful to provide non-human transgenic animals to assay in vivo Dll4 protein function, including ligand interaction, the effect of specific mutant Dll4 proteins on Dll4 protein function and ligand interaction, and the effect of chimeric Dll4 proteins. It is also possible to assess the effect of null mutations, e.g., mutations that substantially or completely eliminate one or more Dll4 protein functions.

### SPECIFIC EMBODIMENTS

As described below, mice were generated in which the Dll4 gene was replaced with a reporter gene. It found that Dll4 expression is initially restricted to large arteries in the embryo, whereas in adult mice and tumor models, Dll4 is specifically expressed in smaller arteries and microvessels, with a striking break in expression just as capillaries merge into venules. Consistent with these arterial-specific expression patterns, heterozygous deletion of Dll4 resulted in prominent albeit variable defects in arterial development (reminiscent of those in Notch knock-outs), including abnormal stenosis and atresia of the aorta, defective arterial branching from the aorta, and even arterial regression, with occasional extension of the defects to the venous circulation; also noted was gross enlargement of the pericardial sac and failure to remodel the yolk sac vasculature. These striking phenotypes resulting from heterozygous deletion of Dll4 indicate that vascular development may be as sensitive to subtle changes in Dll4 dosage as it is to subtle changes in VEGF dosage, as VEGF accounts for the only other example of haploid insufficiency resulting in obvious vascular abnormalities.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Targeting the Dll4 Gene in Mice

Recently described Velocigene™ technology (Valenzuela et al. (2003) Nat. Biotechnol. 21:652-9) was used to generate a precise deletion and exchange of the Dll4 coding region, extending from the initiation to the termination codon (corresponding an 8.1 kB region comprising all of the coding exons and intervening introns), with the beta-galactosidase reporter gene as well as a neomycin selection cassette. Briefly, a bacterial artificial chromosome (BAC) containing the 8.1 kb Dll4 coding region and 140 Kb of flanking sequences (clone 475d4 from a 129/SvJ BAC library obtained from Incyte Genomics) was modified to generate a BAC-based targeting vector which was then linearized and used as a targeting vector to replace the Dll4 gene in F1H4 (C57BL/6:129 hybrid) mouse embryonic stem (ES) cells. Correctly targeted embryonic stem cells were identified using the loss of native allele (LONA) assay (Valenzuela et al. (2003) supra). Two independent correctly targeted ES lines were used to generate chimeric male mice that were complete transmitters of ES-derived sperm. Chimeras were then bred to C57BL6 and/or ICR females to generate F1 mice or embryos, which were genotyped by LONA assays and b-galactosidase histochemical assays. Mice derived from both ES lines behaved identically, and pooled data from both clones were used for statistics.

Tumor implantations. Lewis lung carcinoma cells (ATCC) were subcutaneously implanted into the flank of Dll4 chimeric mice, harvested after 16 days, cut into 80 micron sections, and stained for CD31/PECAM or b-galactosidase as described (Holash et al. (2002) Proc Natl. Acad. Sci. USA 99:11393-8).

PECAM and Reporter staining. Staining of whole-mounted embryos, as well as tissue sections from embryos and adults, were performed as previously described for CD31/PECAM to define the vascular endothelium and for β-galactosidase to visualize the Dll4 reporter gene product (Gale et al. (2002) Dev. Cell 3:411-23).

Quantitative RT-PCR analysis for Dll4, Hey1 and Hes1. The RT-PCR analysis was performed as described (Livak et al. (2001) Methods 24:402-8). The results are expressed as the ratio of the amount of the RNA of interest to the amount of control RNA (GAPDH) as described (Daly et al. (2004) Genes Dev. 18:1060-71) on an Applied Biosystems 7900HT using specific primers and probes as follows: Dll4 Primers: Dll4-1574F: GAGGTCCAAGCCGAACCTG (SEQ ID NO:9) and Dll4-1644R: ATCGCTGATGTGCAGTTCACA (SEQ ID NO:10) and Dll4 Probe: Dll4-1594T: CGCTGCCGGCCTGGATTCAC (SEQ ID NO:11); Hey1 Primers: mHEY1-139F: CAAGCCCGGAAGAAGCG (SEQ ID NO:12) and mHEY1-219R: TCGTCGCAATTCAGAAAGGC (SEQ ID NO:13) and Hey1 Probe: mHEY1-173T: AACGGCGCAGAGACCGCATCA (SEQ ID NO:14); and mHes1 (ID Mm00468601 m1, Hes1 (ABI, Assay on demand services). cDNAs were derived from 6 WT and 3 Het embryos.

### Example 2

### Dll4 Antagonist Fusion Proteins

Example of fusion proteins capable of binding and inhibiting Dll4 include the following constructs. The constructs include the extracellular ligand-binding portion of Notch receptors fused to an oligomerizing domain, in this case human Fc region from IgG. The nucleic acid (cDNA) and amino acid sequences of human Notch1-Fc and mouse Notch1-Fc constructs are shown (SEQ ID NO:15-16 cDNA and amino acid sequence of mNotch1-Fc construct; SEQ ID NO:17 is human Notch1-Fc cDNA).

### Example 3

### Assay for In Vivo and In Vitro Determination of Dll4 Antagonist Activity

The ability of an agent to inhibit Dll4 activity is tested by looking at the effect on tumor blood vessel growth in mice. For example, in the case of a protein reagent, tumor cells are engineered to express the putative Dll4 antagonist, such as Notch1-Fc, and then implanted into immunodeficient mice such as SCID CB17 (from Taconic Farms). Tumor cells engineered to express a non-active protein and implanted into mice serve as controls. Tumors are allowed to grow in the mice until reaching a suitable size, and then the tumors are harvested and stained immunohistochemically for the tumor blood vessels. The blood vessel density and branching are compared in tumors expressing Dll4 antagonist to those expressing non-active control protein.

To assess the in vitro activity of protein-based Dll4 antagonists, the agent is first purified from a suitable cell-based expression system. The purified protein agent is then given in combination with Dll4 agonists to cells that express Dll4.

What is claimed is:

1. A method for identifying an agent capable of binding a Dll4 protein, or protein fragment, comprising:
(a) contacting a test agent with a Dll4 protein, or protein fragment; and
(b) determining the ability of the test agent to bind Dll4 protein or protein fragment.

2. The method of claim 1, wherein the Dll4 protein comprises the amino acid sequence of SEQ ID NO:1 or 3.

3. A method for identifying an agent capable of inhibiting Dll4 activity, comprising:
(a) administering a test agent to an animal expressing a Dll4 protein, or protein fragment; and
(b) determining the ability of the test agent to inhibit Dll4 activity.

4. The method of claim 3, wherein the agent is capable of inhibiting blood vessel formation.

5. The method of claim 4, wherein the agent is an antibody to Dll4.

6. The antibody of claim 4, which is humanized, chimeric, or fully human.

7. The method of claim 4, wherein the agent is a modified Dll4 molecule capable of binding a Notch 1 or Notch 4 receptor without activating said receptor.

8. The method of claim 7, wherein the agent is an antisense or siRNA molecule capable of interfering with the expression of Dll4.

9. A therapeutic method for inhibiting blood vessel development or growth, comprising administering the antibody of claim 5 to a patient in need thereof.

10. An antibody capable of blocking human Dll4 binding activity.

11. The antibody of claim 10, which is humanized, chimeric, or fully human.
